(19) United States
(12) Patent Application Publication
(10) Pub. No.: US 2010/0256713 A1
(43) Pub. Date: Oct. 7, 2010

(54) DEVICES AND METHODS FOR TREATMENT OF HOLLOW ORGANS

(75) Inventors: Stuart D. Edwards, Salinas, CA (US); Peter H. Muller, Woodside, CA (US); Thomas C. Wehman, Cupertino, CA (US)

Correspondence Address: JAMES EPPA HITE, III, 2318 LOUIS ROAD, PALO ALTO, CA, 94303-3635, US

(73) Assignee: Stuart D. Edwards, Prunedale, CA (US)

(21) Appl. No.: 12/144,575

(22) Filed: Jun. 23, 2008

Related U.S. Application Data
(63) Continuation-in-part of application No. 12/108,499, filed on Apr. 23, 2008.
Application No. 12/108,499 is a continuation-in-part of application No. 12/099,349, filed on Apr. 8, 2008, now abandoned.

(51) Int. Cl. A61F 7/12

(52) U.S. Cl. 607/105

(57) ABSTRACT

Minimally invasive apparatus and method for treating medical conditions of hollow organs. The treatment apparatus and method provide for delivery of energy from fluid sources of energy to the interior surface of the hollow organ in contact with the underlying glands, nerves, and muscle walls of the organ.

### RELATED APPLICATIONS

The present application is a continuation in part of U.S. patent application Ser. No. 12/108,499, filed on Apr. 23, 2008, by Edwards et. al., entitled “Treating Medical Conditions of Hollow Organs,” which is a continuation in part of U.S. patent application Ser. No. 12/099,349, filed on Apr. 8, 2008, by Edwards et. al., entitled “Treating Medical Conditions of Hollow Organs;” both applications assigned to the assignee of the present application; the full disclosures of which are incorporated herein by reference.

### FIELD OF THE INVENTION

The present invention is generally related to devices and methods for treating medical conditions of hollow organs, and more particularly, and by way of example, to devices and methods for treating the hollow organs of the digestive system to treat body-weight related conditions.

All patents and published patent applications referred to herein are incorporated herein by reference in their entirety.

### BACKGROUND

The human body has several organs that are considered hollow, such as but not limited to: organs of the GI Tract (e.g., esophagus, stomach, small and large intestines), bladder, ear canal, nasal sinuses, female reproductive system (e.g., vagina, vaginal canal, uterus, fallopian tubes) and the lungs.

Each of these and other hollow organs can be subject to medical conditions such as cancer or conditions resulting from loosening of the muscles underderlying the organ. Treatment for these medical conditions range from pharmaceutical therapies to highly invasive surgeries.

As an example, obesity is one major medical condition that affects several hollow organs of the GI Tract. Obesity is directly associated with other medical disorders, such as: osteoarthritis (especially in the hips), sciatica, varicose veins, thromboembolism, ventral and hiatal hernias, hypertension, insulin resistance, and hyperinsulinemia; premature death; type 2 diabetes, heart disease, stroke, hypertension, gall bladder disease, GI tract cancers, incontinence, psychological disorders, sleep apnea, gastro esophageal reflux disease (GERD), and liver disease. Reducing obesity reduces the effects of these conditions provided the weight loss is significant and enduring. This, of course, is the challenge to the patient and practitioner.

Current obesity treatments include behavior modification, pharmaceutical interventions, and invasive surgeries.

One problem with behavior modification is patient compliance. Significant and maintained weight loss demand enormous levels of patient compliance over a long time.

Problems with pharmaceutical intervention include drug dependence and adverse side effects. Amphetamine analog treatments involve habitual use of addictive drugs to produce and maintain significant weight loss. Dexfenfluramine and fenfluramine treatments often result in primary pulmonary hypertension and cardiac valve abnormalities. Drugs such as sibutramine substantially increase blood pressure in many patients.

Surgical obesity treatments include invasive surgical procedures such as: gastric banding, bariatric surgery, and liposuction. While current surgical procedures can be effective, the overall rates of surgical mortality and associated hepatic dysfunction are so high that surgical treatments are only indicated for younger patients who are morbidly obese.

The following table outlines various conventional treatments for obesity and issues associated therewith:

| Treatment | Issues |
|---|---|
| Diet, Exercise and | 90% unsuccessful |
| Behavior | |
| Modification | |
| Pharmacotherapy | Moderate benefits and risk of |
| | dependence |
| Very Low Calorie | Patients of regain weight |
| Diet and Medically | |
| Supervised Programs | |
| Gastric Banding | Invasive, risks, complications, |
| | long-term care, costly |
| Bariatric Surgery | Invasive, risks, complications, |
| | long-term care, costly |
| Liposuction | Invasive, risks, complications, |
| | long-term care, costly |

U.S. Pat. No. 7,326,207 proposes treating obesity by mapping (for example, using a visualization apparatus, such as but not limited to endoscopes or fluoroscopes) and ablating nerves in targeted stomach areas by creating patterns of thermal lesions. The nerves are ablated using surface electrodes that penetrate the nerves during energy application. Mapping is required to properly position the electrodes where they can penetrate the nerves. Physiological changes caused by tissue ablation create a sense of satiety in the patient by directly modulating nerves responsible for hunger sensation or by modulating the nerves inhibiting the let-down reflex of the stomach muscles that are digestion precursors.

Despite the treatment described in the '207 patent, there is room for further improvement in the field of obesity treatment and as well treatment of other medical conditions that affect hollow organs.

### SUMMARY OF THE INVENTION

The present invention relates to devices and methods for treatment of hollow organs. In an embodiment, the present invention relates to devices and methods for treatment of the digestive system, such as the stomach, for weight-related conditions. The present devices, assemblies, and methods apply energy to, among other things, any one or more of muscles, nerves, and glands associated with and/or underlying the hollow organ to alter any one or more of the muscular profile of the organ, its biomechanical operation, or physiological properties. In an embodiment, energy is applied by way of surface contacts that can be easily positioned to, directly or indirectly, apply energy to any one or more of the surfaces of nerve branches, muscles, or glands associated with the communication paths between the hollow organ and the brain. The nerves, muscles, and/or glands are exposed to a source of energy by expanding the organ beyond its normal volume until the organ mucosa is separated and the underlying nerves, muscles, and/or glands are exposable to the energy. In an embodiment, treatments embodying features of the present invention enable the modification of any one or more of the nerve signal transmission, muscle profile to a profile more suitable for reaching treatment goals, or the gland's enzyme release.

An apparatus for treating medical conditions of hollow organs embodying features of the present invention may include a hollow organ treating assembly having an organ expanding member configured for expansion in the hollow organ to expose at least a portion of either or both the hollow organ's underlying nerves and muscle. The organ expanding member is configured to conform the organ's volume to that of the organ expanding member in the expanded configuration. In an exemplary configuration, the organ expanding member, such as an expandable balloon, is disposed at a distal end of an elongate expanding body such as a catheter. The organ treatment assembly further includes an energy delivery assembly including an energy delivery member disposable at a distal end of an elongate member. The energy delivery member is disposable within an inner space of the organ expanding member for delivering energy to the organ expanding member and thus to the organ. The energy delivery elongate member includes at least one lumen for delivering energy, such as hot fluids, to and from the energy delivery member. In an embodiment, the energy is in the form of a hot fluid such as steam, hot water, hot saline, and the like. Contact surfaces along at least a portion of an outer surface of the organ expanding member provide for at least partial direct surface contact with the inner surface of the hollow organ. In an embodiment, corresponding inner surfaces of the organ expanding member are configured for at least partial direct contact with at least a portion of the energy delivery member. Energy is transferrable, at least in part, from the energy delivery member to the organ expanding member, and to the inner surface of the hollow organ.

The organ expanding member includes contact surfaces which are associatable with select portions of the hollow organ such as the stomach. The associatable contact surfaces of the organ expanding member typically correspond to at least one of the greater curvature of the stomach, smaller curvature of the stomach, cardiac zone, gastric/fundic zone, pyloric zone, or the vagal nerve associated with the stomach. A distal portion of the organ expanding member may be shaped to conform, upon expansion, to the pyloric sphincter. The expanded distal portion of the organ expanding member, during operation, seats against the distal end of the pyloric sphincter and aids in the positioning of the organ expanding assembly.

The treatment assembly may further include an endoscope disposable within the elongate expanding body. The endoscope may include one or more inflation/deflation lumens and a lumen for receiving the energy delivery assembly. The energy delivery member, in an unexpanded state, seats at the distal end and within the endoscope lumen in which it is received. The energy delivery member is configured to, in use, extend out of the distal end of endoscope in order to treat the desired area of the organ.

The Endoscope may include an illumination source such as lights for visualization of the organ when it is disposed within an organ's interior. In an embodiment, the endoscope, with the energy delivery assembly disposed within its lumen, is disposed within the organ expanding assembly prior to advancement into the organ. The organ expanding assembly, the endoscope, and the energy delivery assembly, are accessible through a hand-piece disposable at the proximal end of the organ treatment assembly.

The energy delivery member is configured for fluid communication with an energy source. The energy source, such a thermal energy source, may include, but is not limited to, hot fluids such as hot water, hot saline, hot air, steam; and is controllable by the control assembly. The temperature of the energy at its source is sufficiently high to provide a sufficiently high fluid temperature at the point of treatment. The temperature of the energy at its source may range from about 60 to about 110° C., from about 70 to about 105° C., from about 75 to about 85° C. The temperature at the point of treatment is sufficiently high to effectuate the desired treatment. The temperature at the point of treatment may range from about 50 to about 100° C., from about 60 to about 95° C., from about 60 to about 80° C. One or more sensors may be located within or about the energy delivery member. In an embodiment, the sensor is a thermocouple for sensing the temperature, as for example, when hot water is delivered to an interior of the energy delivery member. It should be appreciated that the sensor may take any appropriate form, as for example formed of wireless construction, and may further be configured to sense and convey the necessary information in any number of ways and formats and is not limited to direct thermal sensors. By way of example and not limitation, the temperature may be sensed by optical means which can assess a change in the color of a portion of member. In this configuration, colorants may be present in the material forming the member or be painted or deposited on its material (on the inner or outer surface). Once the colored area is exposed to the elevated temperature, the colorant may change its characteristics. Information as to the temperature may then be conveyed to the practitioner. The information may be conveyed automatically by instrumentation or by direct visualization through the endoscopic device. Additionally or alternatively, such sensors may also be present on or in the organ expanding member.

The organ expanding member and the energy delivery member may, independently, be made from any suitable material such as, but not limited to expandable, noncompliant (or semi-compliant) material including Mylar, Nylon, PET, PeBax, IEBA. In a preferred embodiment, the material for balloons is formed from non-compliant material. For example, Mylar, while expandable, is noncompliant and restricts expansion of the expandable balloon within the stomach. Therefore, an expandable balloon formed from Mylar cannot infinitely expand and patient injury resulting from unintended over-inflation of expandable balloon can be reduced. In an embodiment, when used for obesity treatment, the expandable balloon is constructed such that when inflated within the stomach, the stomach expands from its empty volume (about 1 liter) to at least about twice the stomach's empty volume (e.g. 2 liters). However, for other organs and other species, the organ expanding member may have different profiles or volumes. In an embodiment, the expanding member is pre-shaped such that as the expanding member is expanded within the organ, the organ's interior conforms to the profile of the expanding member. As noted earlier, the energy delivery member may also be pre-shaped.

Optional visual markings, corresponding to desired target areas of the organ, may be located on either or both the organ expanding member or the energy delivery member. The visual markings are used to aid in locating the desired treatment target areas. Such visual markings may be incorporated into or deposited on or within the material forming the member. In an embodiment, the visual markings may take the form of colorant, metallic or polymeric material. Although some sort of visual marking may be preferred, the practitioner may identify the necessary areas for transfer of energy using practitioner's experience. The visual markings may be positioned to correspond to any one or more areas corresponding to or in the vicinities of the greater curvature of the stomach, smaller curvature of the stomach, cardiac zone, gastric/fundic zone, pyloric zone, or the vagal nerve within the stomach.

In an embodiment, the treatment assembly further includes an external treatment controller for controlling various parameters useful in the treatment procedure. The external treatment controller includes any one or more of the following subassemblies: treatment energy source for providing source of treatment energy such as a fluid source (e.g., steam, hot water, hot saline), input/output (I/O) device, inflation fluid delivery unit for inflating/deflating the organ expanding member and/or the energy delivery member, and GUI.

In an exemplary method for treating the hollow organ embodying features of the present invention includes using a source of energy positionable in the organ to apply energy to at least one surface of the organ to affect the organ's operation. In an embodiment, the energy, is thermal energy provided from a source of hot fluid, such as steam, hot water, or hot saline. An exemplary hollow organ is the stomach and the at least one surface of the organ to which energy is applied is either or both stomach's muscle surface or the surface in the vicinity of at least a portion of a nerve communicating with the stomach and brain. In an embodiment, the treatment includes applying energy to at least one surface of the stomach's underlying glands to affect glandular emissions, such as ghrelin, pepsin, rennin, and/or HCl. In an exemplary method, the treatment includes transferring energy from an energy delivery member, positionable within the organ, by way of, direct or indirect, surface contact. The energy may be transferred from the energy delivery member to the organ by way of surface contact with an organ expanding member disposable between the energy delivery member and the hollow organ's inner surface.

In an exemplary embodiment, a method for treating the hollow organ, such as the stomach in the digestive tract, includes introducing an organ expanding member into the hollow organ; expanding the hollow organ with the organ expanding member to bring into surface contact at least a portion of a surface of the organ expanding member with at least a portion of a surface of the hollow organ to expose any one or more of the underlying nerves, muscles, or glands of the organ. A source of hot fluid is controllably introduced into an interior of the organ expanding member and thermal energy from the hot fluid source is delivered to at least a portion of the surface of the organ expanding member and to at least a portion of the surface of the hollow organ. At least one or more of underlying nerve, muscle, or gland of the stomach are treated (e.g., ablated). In an embodiment, the at least one or more of underlying muscle is at least in one of the greater curvature of the stomach, smaller curvature of the stomach, cardiac zone, gastric/fundic zone, pyloric zone, or the vagal nerve within the stomach.

In an exemplary method of treatment of the stomach, a gastric introducer is positioned in the patient's throat. The expanding assembly, preloaded with the endoscope and the energy delivery assembly, is inserted into patient's body through the introducer. The expanding balloon is advanced distally positioning its shaped distal portion against the distal side of the pyloric sphincter. The organ expanding member is inflated until the stomach's volume reaches the desired volume, such as at least about twice its empty volume (e.g. to about 2 liters). The inflation fluid for expansion of the expanding member may be by way of the inflation/deflation lumen of the endoscope. Alternatively, the expanding elongate body may have an inflation/deflation lumen itself usable for delivery and/or removal of the inflation fluid which is in fluid communication with the inflation fluid source and the organ expanding member. The expanded pre-shaped distal portion of the expanding member is seated against the distal side of the pyloric sphincter, providing an anchor and aiding in position and placement of the organ expanding assembly within the organ. In an exemplary embodiment, inflation fluid is delivered to the organ expanding member through the inflation/deflation lumen of the endoscope.

The endoscope with the energy delivery member disposed within it, is pulled back proximally and navigated to the desired areas within the organ. In an exemplary method, the visual markings on the organ expanding member are used to aid in the placement of the endoscope.

Hot fluid, such as hot water from the hot fluid source, is introduced into the interior of the energy delivery member using the inflation/deflation lumen of the energy delivery elongate member. The energy delivery member is extended out of the distal end of the endoscope and into the interior of the organ expanding member. It should be noted that the delivery of hot fluid to the energy delivery member and the extending of the energy member from the distal end of the endoscope may be performed before, simultaneous, or after the navigation of the endoscope and/or energy member within the expanding member. It should be appreciated that the delivery and the removal of the energy fluid may be achieved by way of other suitable lumens in fluid communication with the energy fluid source and the energy delivery member as for example

Under visual guidance by way of the endoscope, the expanded energy delivery member is directed toward interior surface of the organ expanding member corresponding to the desired treatment area/s of the target organ. Energy (e.g., thermal energy from the hot fluid) is directed to or the vicinities of the nerves, muscles and glands of the treatment zones. The desired treatment area/s may include any one or more areas corresponding to or in the vicinities of the greater curvature of the stomach, smaller curvature of the stomach, cardiac zone, gastric/fundic zone, pyloric zone, or the vagal nerve within the stomach.

In an embodiment, the expanded energy delivery member is brought into direct surface contact with the desired surface area of the organ expanding member which corresponds to the desired target areas. Energy is delivered from the energy delivery member to the organ expanding member which is in direct surface contact with the desired treatment sites.

In an embodiment, the transferred energy is sufficiently high to cause a change (e.g., physical, biochemical, physiological change) in the treatment target areas as described earlier.

Upon reaching a desired level of tissue treatment, the energy delivery member and organ expanding member are deflated and are withdrawn from the patient, followed by the removal of the gastro introducer.

### BRIEF DESCRIPTION OF THE DRAWINGS

FIGS. 1A and 1B are simplified illustrations of a mammalian digestive system.

FIG. 2A illustrates an exemplary stomach treatment assembly of an apparatus embodying features of the present invention for treating body-weight related conditions.

FIGS. 2AA-AC are cross sectional views of portions of the organ expanding assembly, endoscope, and the introducer of FIG. 2A.

FIGS. 2B-2C are schematic and cross sectional views of portions of the endoscope of FIG. 2A with an energy delivery assembly disposed therein.

FIGS. 2D and 2DA are schematic and cross sectional views of an embodiment of a portion of the energy delivery assembly.

FIGS. 2E and 2F are schematic and cross sectional views of alternate embodiments of portions of the organ expanding assembly.

FIG. 3 illustrates a schematic of an exemplary external control unit embodying features of the present invention for use with the stomach treatment assembly of FIG. 2A.

FIGS. 4A-G illustrate various steps of an exemplary therapeutic procedure embodying features of the present invention.

FIGS. 5A and 5B are schematic representations of neural communication between the stomach and the brain.

FIG. 6 illustrates a schematic profile of a treated stomach about 8-12 weeks, post-op.

### DETAILED DESCRIPTION OF THE PREFERRED EMBODIMENTS

### Anatomical Background

In describing features of the present invention, the hollow organ of the digestive system, such as the stomach, will be used. However, it should be appreciated by those skilled in the art that the use of this exemplary organ is not intended to limit the scope of the present invention.

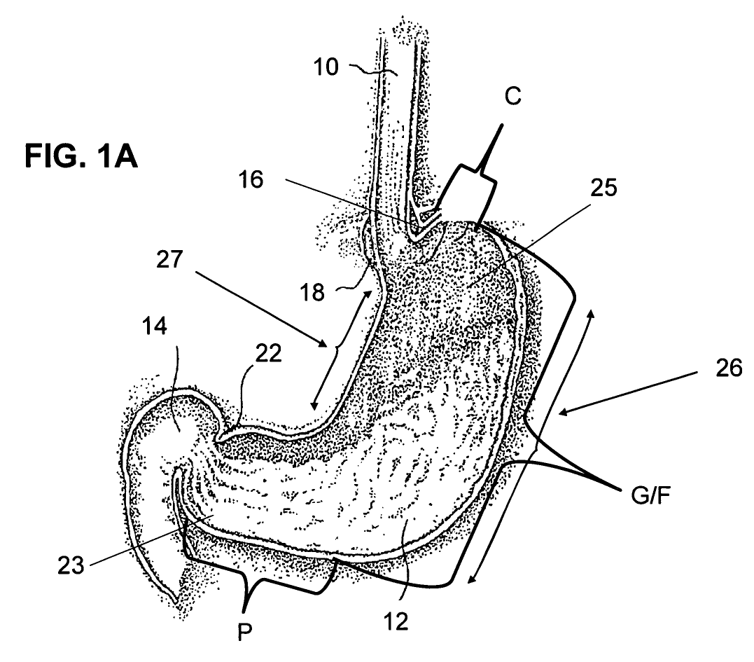

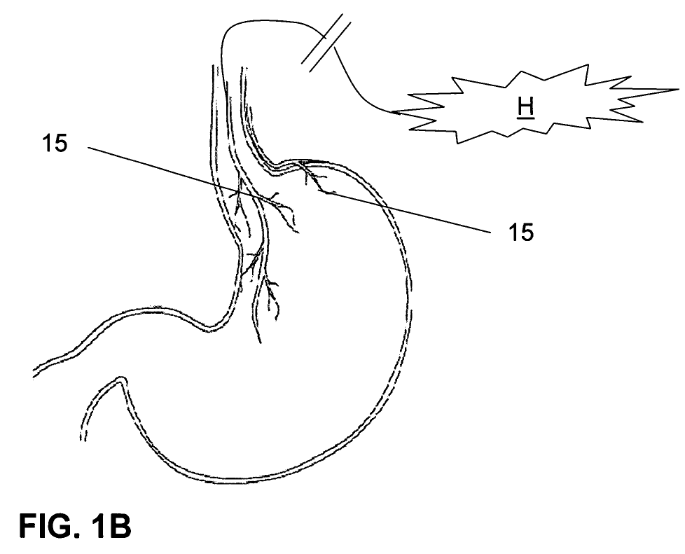

FIGS. 1A and 1B are simplified depictions of a mammalian digestive system. These FIGS. are not intended to be strictly accurate in an anatomic sense or imply that the teachings of this patent application are limited strictly to treating the digestive system. The drawings show the digestive system in somewhat diagrammatic form for purposes of discussion.

FIG. 1A, illustrates esophagus 10, a muscular tube, for carrying food from the mouth to the stomach 12, by way of wavelike contractions of the muscles in the walls of the esophagus 10. The interior esophagus walls include glands that secrete mucus, which further aid the movement of food by acting as lubricants.

Stomach 12, located in the upper left hand side of the abdomen, lies between the esophagus 10 and the small intestine 14. In humans and most other animals, stomach 12 is a simple baglike organ.

FIG. 1B depicts branches 15 of the vagal nerve that connect stomach 12 with the hindbrain H which is believed to be the neurological source for the hunger sensation. The upper end of stomach 12 connects with the esophagus 10 at cardiac notch 16 (FIG. 1A). The muscular ring called the lower esophageal sphincter 18 surrounds the opening between the esophagus 10 and the stomach 12. The funnel-shaped region of the stomach 12 immediately next to sphincter 18 is the cardia. The cardia (also known as Z-line or esophagogastric junction or gastroesophageal junction) is the anatomical term for the junction orifice of the stomach and the esophagus. At the cardia, the mucosa of the esophagus transitions into gastric mucosa. The cardia is also the site of the lower esophageal sphincter 18 (LES which is also termed cardiac sphincter). The greater curvature of the stomach, 26, starts from the cardiac orifice at the cardiac notch, and forms an arch backward, upward, and to the left; the highest point of the convexity is on a level with the sixth left costal cartilage. The lesser curvature 27 of the stomach is opposite the greater curvature and extends between the cardiac and pyloric orifices, forming the right or posterior border of the stomach. It descends as a continuation of the right margin of the esophagus in front of the fibers of the right crus of the diaphragm, and then, turning to the right, it crosses the first lumbar vertebra and ends at the pylorus.

From this level it may be followed downward and forward, with a slight convexity to the left as low as the cartilage of the ninth rib; it then turns to the right, to the end of the pylorus. Positioned below the cardia is the fundus 25 of the stomach.

The volume of an average adult stomach, an organ for storing and digesting food, is a little over one quart (˜0.95 liter). Pyloric sphincter 22, located distal of pylorus 23, surrounds and controls the size of the duodenal opening disposed between stomach 12 and small intestine 14. Pyloric sphincter 22 keeps non-liquid food in stomach 12 until the food is processed into a more flowable liquid form, thereafter allowing for the flow of the liquefied food from stomach 12 into the intestine 14. The time food spends in stomach 12 varies and usually ranges from about three to about five hours.

Using these anatomical features as landmarks or guides, the human stomach is often described as having three zones, namely: cardiac zone, gastric/fundic zone, and pyloric zone. In an embodiment, A treatment according to the body-weight related conditions, according to the present invention, is achieved by applying energy to or in the vicinities of any one or more of:

(1) nerve tissue which allows nerve pulse communication between the hindbrain H and stomach 12; or

(2) stomach tissue to ablate tissue in one or more areas where food is either processed and/or absorbed by the body, for example, the cardiac, gastric/fundic, and pyloric zones.

Additionally, treatment may be expanded to other areas, such as the small intestine (and associated nerves), where about 95% of all food absorption occurs. Ablation, or causing cell death, produces lesions which when large enough, evoke tissue-healing and intervention of fibroblasts, myofibroblasts, macrophages, and other cells. Healing results in tissue contraction (shrinkage), decreased volume, and/or altered biomechanical properties. In contrast with other treatments for conditions such as obesity which merely try to prolong patient satiety, the current devices and methods embodying features of the present invention, further provide for directly affecting the digestive process and may reduce food absorption. Without intending any limitations on the scope of the present invention, it is believed by the present inventors that ablation of cells in the cardiac, gastric/fundic, and/or pyloric zones enables treatment of weight-related conditions and reduces a patient's body weight, among other things, for the following reasons:

CARDIAC AND FUNDIC-GASTRIC ZONES—The cardia and fundic-gastric zone contain, respectively, the cardiac glands (not shown) and the fundic glands (not shown). The cardiac and fundic glands release digestive enzymes (e.g., ghrelin, pepsin and rennin) and hydrochloric acid (HCl) which are used during digestion to break down food. Ablating a portion of the cardiac and gastric-fundic zones, e.g., the cardiac and fundic glands, therefore, reduces the release of ghrelin, pepsin, rennin and HCl, thereby reducing the amount of food digested by the body and resulting in more undigested food particles passing through the patient's body.

PYLORIC ZONE—The pyloric sphincter controls the size of food particles and their flow from the stomach (emptying cycle). The wider the opening of the sphincter, the larger the size of the food particles that may flow out of the stomach. Without limiting the scope of the present invention, it is believed that ablating the pyloric muscle tissue decreases the size of the pyloric opening and the size of food particles that may flow out, thereby lengthening the emptying cycle (longer sensation of satiety).

The gastric zone also includes the lesser curvature of the stomach which contains nerves that control peristalsis of the stomach walls. Peristalsis contributes to digestion by physically reducing the size of food particles in the stomach. It is also believed, without limiting the scope of the present invention, that ablating portions of the muscles of the lesser curvature reduces peristalsis and increases food particle size. These larger food particles, when passed through the pyloric sphincter, cannot be digested through the small intestine and therefore would pass through the patient's body undigested. Finally, ablating gastric zone tissue may also affect the gastric glands and reduce HCl production in the stomach (see above).

Against this anatomical and physiological background, exemplary apparatus, assemblies, and methods for treating body-weight related medical conditions associated with hollow organs will be described. It should be appreciated by those skilled in the art that using the digestive hollow organ, such as the stomach, for describing features of the present invention, is not intended to limit the scope of the present invention.

### Treatment Apparatus

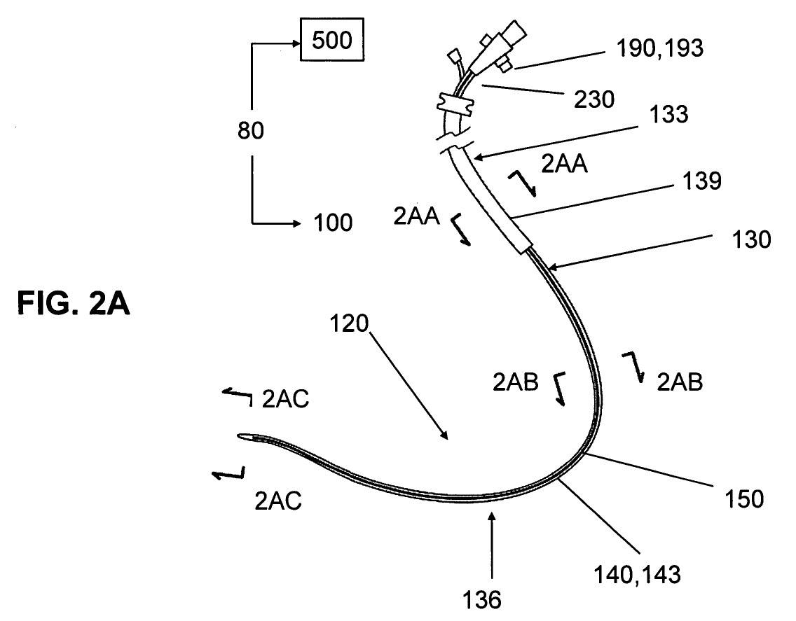

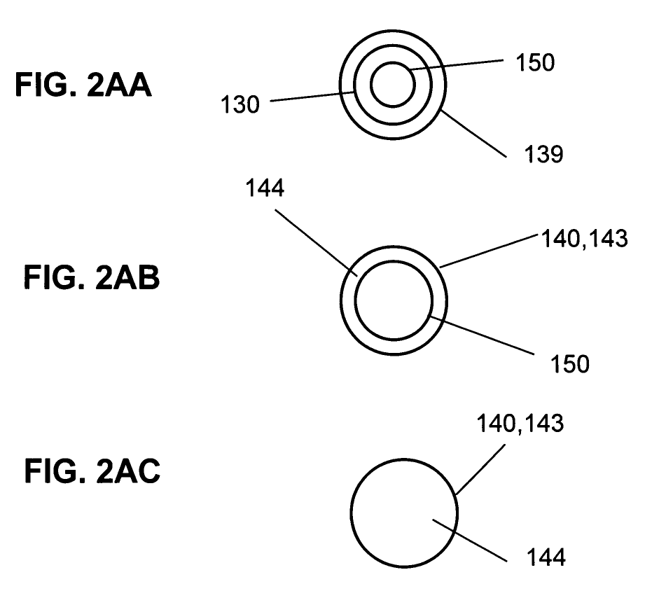

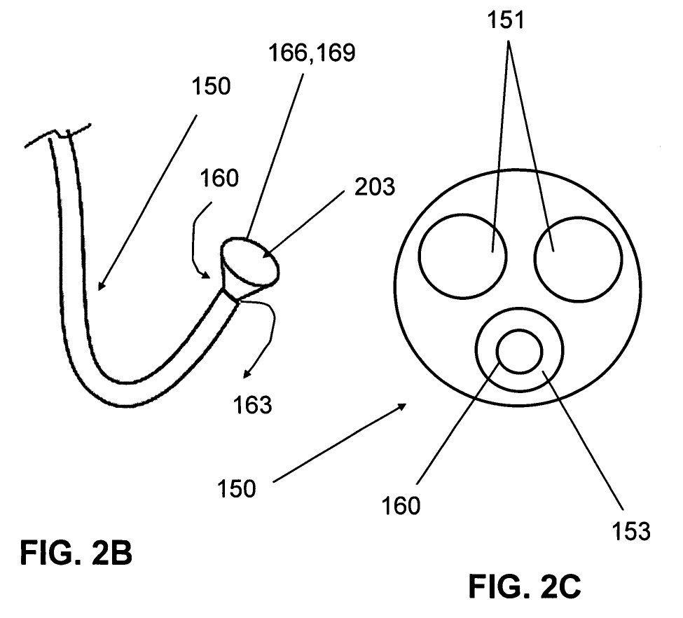

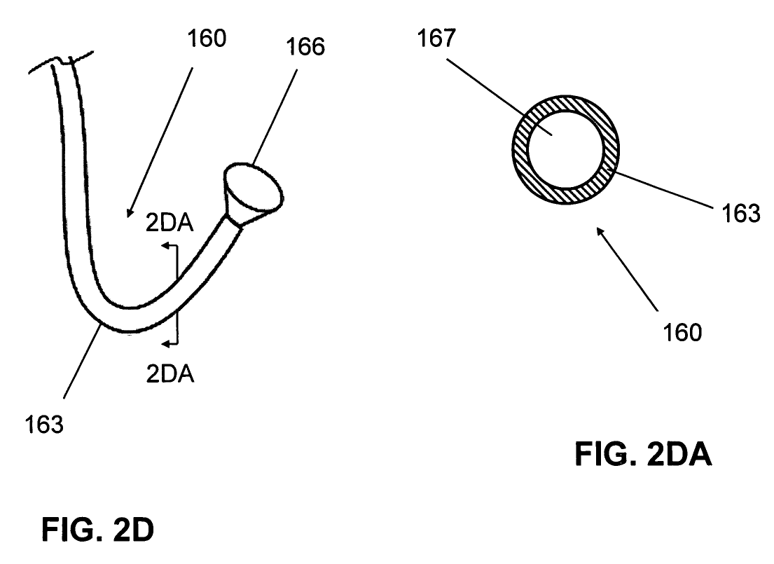

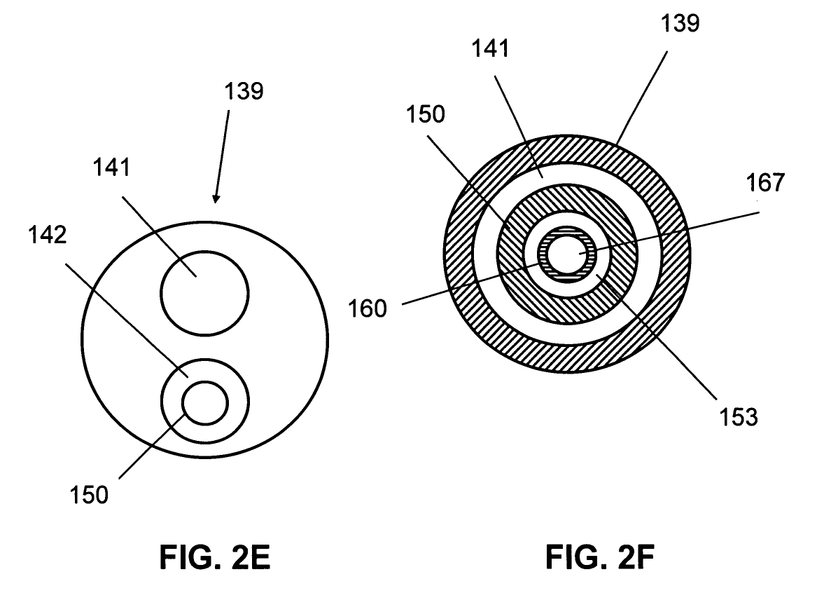

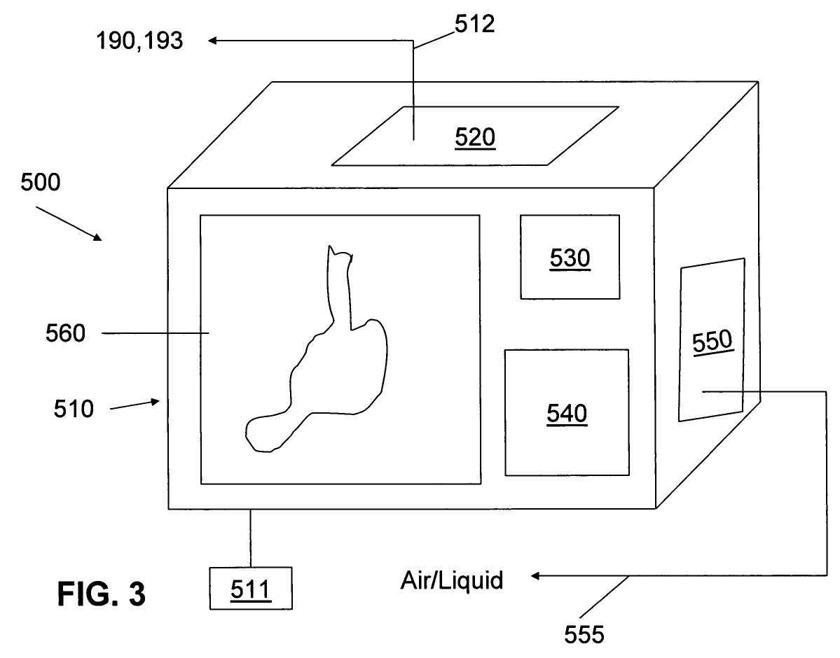

FIGS. 2A through 3 show features of an exemplary embodiment of apparatus 80 for treating hollow body organs.

Assembly 80 includes an organ treating assembly 100 (FIG. 2A) and an external control assembly 500 (FIG. 3). At least a portion of the organ treating assembly 100 works inside the patient's body for treatment of the hollow organ such as digestive tract, e.g., stomach 12 (see FIGS. 1A and 1B). External control assembly 500 includes components for, among other things, controlling, monitoring, and viewing, at least parts of the organ treating assembly 100.

The organ treating assembly 100 includes an organ expanding assembly 120 having an expanding elongate body 130 with proximal and distal ends, 133 and 136, respectively, and at least one lumen, such as lumen 141 (FIGS. 2E-2F) extending along at least a distal portion thereof. As shown, the organ expanding assembly is extending from a distal end of an introducer 139. An organ expanding member 140, such as an expandable balloon 143 having an interior 144, is disposed at the distal end 136 of the organ expanding elongate body 130. As shown in FIG. 2A and cross-sections 2AA and 2AB, an endoscope 150 is disposable within the expanding elongate body 130 and interior 144 of the expandable balloon 143. For purposes of clarity, cross sectional FIGS. 2AA-2AC only show the organ expanding assembly and the endoscope.

Now referring to FIGS. 2B-2D and 2DA, a portion of an embodiment of the endoscope 150 is shown (for purposes of clarity in FIG. 2C only the endoscope 150 is shown). Endoscope 150 includes one or more inflation/deflation lumens 151 and a lumen 153 for receiving an energy delivery assembly 160. As shown in FIG. 2B energy delivery assembly 160 includes an energy delivery elongate member 163 with an energy delivery member 166, such as an expandable energy balloon 169, disposed at a distal end thereof. The energy delivery member 166, in an unexpanded state, seats at the distal end and within lumen 153 of endoscope 150. The energy delivery member 166 is configured to, in use, to extend out of the distal end of endoscope 150 in order to treat the desired area of the organ. As shown in FIG. 2DA, the energy delivery elongate member 163 includes at least one lumen 167 for delivering energy, such as hot fluids, to and from the energy delivery member 166. Endoscope 150 may include an illumination source such as lights (not shown) for visualization of the organ when it is disposed within the organ's interior 300. In an embodiment, endoscope 150 with the energy delivery assembly 160 disposed within its lumen is disposed within the organ expanding assembly 120 prior to advancement into the organ. Alternatively, as shown in FIG. 2E, expanding elongate member 139 includes a lumen 141 for delivering inflation fluid to and from expanding member 140, 143; and lumen 142 for receiving endoscope 150 therein. In another alternate embodiment shown in FIG. 2F, expanding elongate member 139 includes lumen 141 for delivering inflation fluid to and from expanding member 140, 143, as well as receiving endoscope 150 therein. Endoscope 150 includes lumen 153 for receiving energy delivery assembly 160 with the energy delivery lumen 167. As can be appreciated by those skilled in the art, multiple configurations for the various components are usable and within the scope of the present invention.

Now referring back to FIG. 2A, the organ expanding assembly, the endoscope, and the energy delivery assembly, are accessible through hand-piece 230 disposed at the proximal end of the organ treatment assembly.

The energy delivery member 166 is configured for fluid communication with an energy source 190. Energy source 190, such a thermal energy source 193, may include, but is not limited to, hot fluids such as hot water, hot saline, hot air, steam; and is controllable by the control assembly 500. The temperature of the energy at its source is sufficiently high to provide a sufficiently high fluid temperature at the point of treatment. The temperature of the energy at its source may range from about 60 to about 110° C., from about 70 to about 105° C., from about 75 to about 85° C. The temperature at the point of treatment is sufficiently high to effectuate the desired treatment. The temperature at the point of treatment may range from about 50 to about 100° C., from about 60 to about 95° C., from about 60 to about 80° C. One or more sensors may be located within or about the energy delivery member 166. In an embodiment, the sensor is a thermocouple for sensing the temperature, as for example, when hot water is delivered to an interior 203 of the energy delivery member 166. It should be appreciated that the sensor may take any appropriate form, as for example formed of wireless construction, and may further be configured to sense and convey the necessary information in any number of ways and formats and is not limited to direct thermal sensors. By way of example and not limitation, the temperature may be sensed by optical means which can assess a change in the color of a portion of member 166. In this configuration, colorants may be present in the material forming the member 166 or be painted or deposited on its material (on the inner or outer surface). Once the colored area is exposed to the elevated temperature, the colorant may change its characteristics. Information as to the temperature may then be conveyed to the practitioner. The information may be conveyed automatically by instrumentation or by direct visualization through the endoscopic device. Additionally or alternatively, such sensors may also be present on or in the organ expanding member.

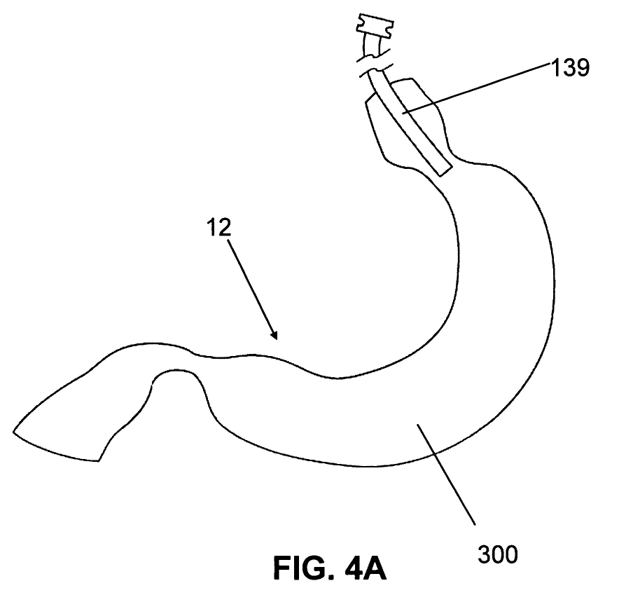

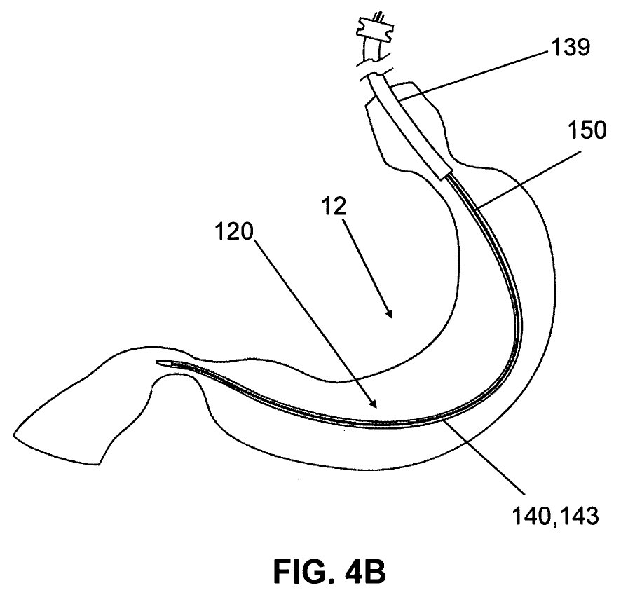

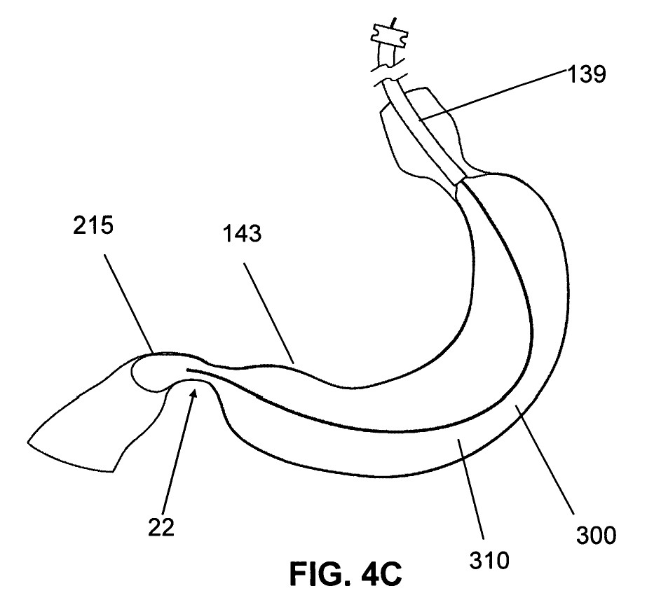

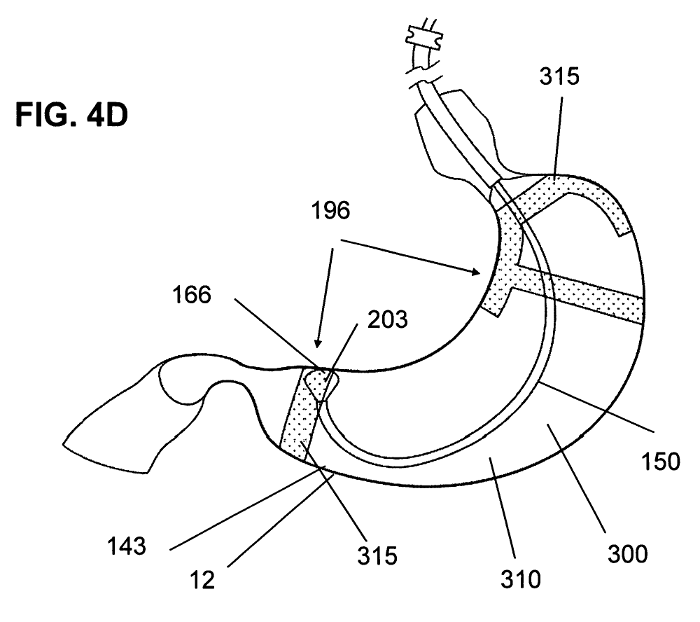

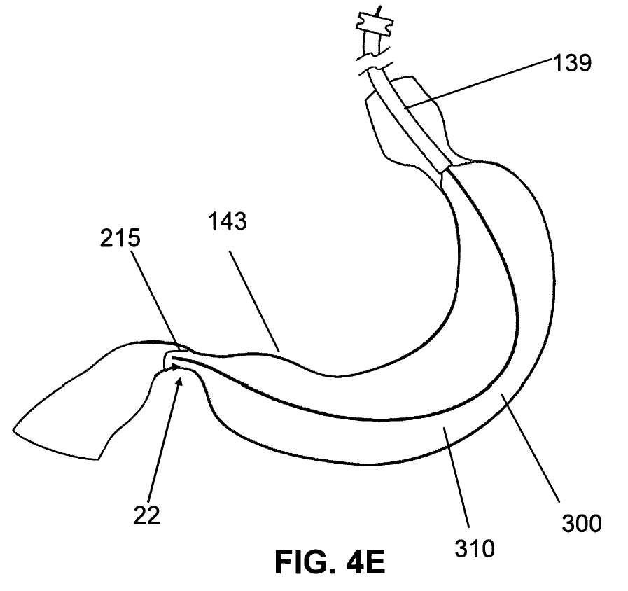

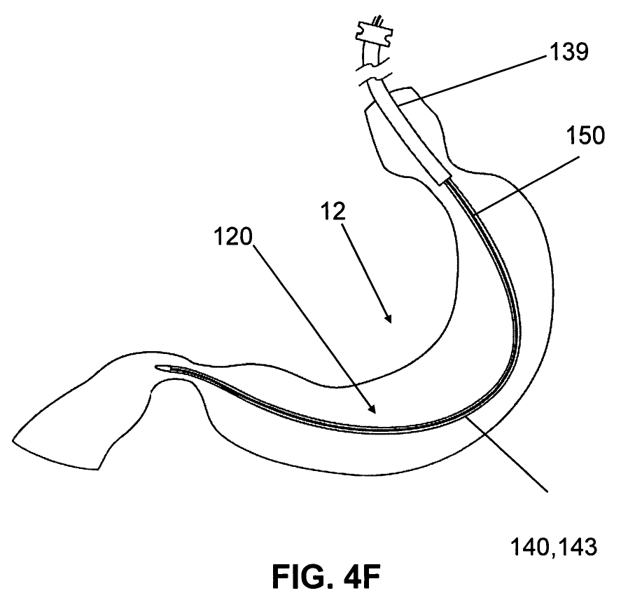

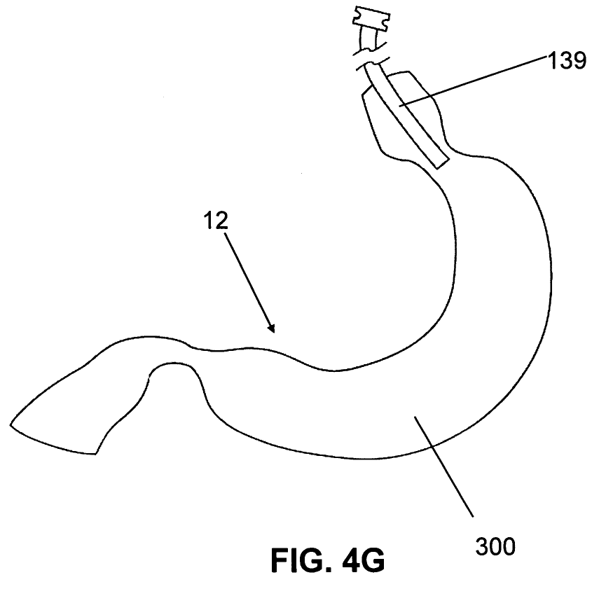

In an embodiment, features of which are further shown in FIG. 4C, a distal portion 215 of the organ expanding balloon 143 is shaped to conform, upon expansion, to the pyloric sphincter 22. In operation the shaped distal portion 215 is inflated and seats against the distal end of the pyloric sphincter. An interior 310 of expanding balloon 143 is in fluid communication with an inflation/deflation source (inflation deflation may be achieved through the same or different lumens) through lumen 151 of the endoscope or lumen 141 of the expanding elongate body 139 (or separate line which may be disposed within lumen 141). Inflation may also be achieved by way of a separate lumen extending along an exterior of the endoscope and disposed within the interior of the expanding assembly 120.

Optionally, visual markings 315 corresponding to desired target areas 196 of the organ and depicted as dotted areas in FIG. 4D, may be located on either or both the organ expanding member or the energy delivery member. The visual markings, are used to aid in locating the desired target treatment areas. Such visual markings may be incorporated into or deposited on or within the material forming the member. In an embodiment, the visual markings may take the form of colorant, metallic or polymeric material. It should be appreciated that the number, shape, location, and configuration of such marking 315 is not limited to those shown. It should further be appreciated that although some sort of visual marking may be preferred, the practitioner may identify the necessary areas for transfer of energy using practitioner's experience. The visual markings 315 may be positioned to correspond to any one or more areas corresponding to or in the vicinities of the greater curvature of the stomach, smaller curvature of the stomach, cardiac zone, gastric/fundic zone, pyloric zone, or the vagal nerve within the stomach.

Regardless of the configuration, organ expanding member 140 and the energy delivery member 166, 169 may, independently, be made from any suitable material such as, but not limited to expandable, noncompliant (or semi-compliant) material including Mylar, Nylon, PET, PeBax, IEBA. In a preferred embodiment, the material for balloons 143 and 169 is formed from non-compliant material. For example, Mylar, while expandable, is noncompliant and restricts expansion of the expandable balloon within the stomach. Therefore, an expandable balloon 143 formed from Mylar cannot infinitely expand and patient injury resulting from unintended over-inflation of expandable balloon 143 can be reduced. In an embodiment, when used for obesity treatment, expandable balloon 143 is constructed such that when inflated within the stomach, the stomach expands from its empty volume (about 1 liter) to at least about twice the stomach's empty volume (e.g. 2 liters). However, for other organs and other species, the organ expanding member 140 may have different profiles or volumes. In an embodiment, the expanding member is pre-shaped such that as the expanding member is expanded within the organ, the organ's interior conforms to the profile of the expanding member. As noted earlier, the energy delivery member may also be pre-shaped.

### External Control

Now referring back to FIGS. 2A 3, the external control portion 500 for apparatus 80 is shown and including a control unit 510. Control unit 510 may include any one or more of the following subassemblies: treatment energy source 520 for providing and controlling fluid source 190, controller 530, I/O device 540, inflation fluid delivery unit 250, and GUI 560.

Summarily, control unit 510 governs the power levels, cycles, and duration of energy transmitted through line 512 to the energy delivery member 166 to achieve and maintain temperature levels that achieve treatment objectives. Foot switch 511 allows hands-free control of energy delivery (e.g., heating and delivery of hot fluid). In tandem, control unit 510 controls delivery of processing (inflation) fluid and, if needed, the removal of aspirated material through fluid lines 555.

Controller 510 includes an Input/Output (I/O) device 540. The I/O device 540 allows practitioners to enter control and processing factors enabling control unit 510 to generate correct command signals. The I/O device 540 also receives real time processing feedback information from the one or more sensors associated with either or both the energy delivery member or the organ expanding member as well as the endoscope (e.g., visualization data). The feedback information is processed by the controller 530, to govern energy application and processing inflation fluid delivery. The I/O device 540 also includes a graphical user interface (GUI) 560 that graphically presents processing information to the practitioner for viewing and/or analysis.

### Therapeutic Procedure/Method

Now referring to FIGS. 4A-4G, various exemplary steps of a therapeutic method are shown. It should be noted, however, that the order of the steps is not necessarily limited to that shown and that they may be altered depending on judgment of the practitioner and the given configuration of the assemblies. Because practitioners need not make any incisions, and in far contrast to the complex and highly invasive bariatric surgeries currently practiced, the treatment according to the present invention is minimally invasive. In an embodiment, the procedure takes about one hour, including preparation and minimal recovery times. In an embodiment, patients can be treated on an out-patient basis using conscious sedation and since the risk of serious problems during the treatment is low it does not necessarily require the complete back-up of a hospital for emergencies.

In an embodiment, and in operation, a gastric introducer 139 is positioned in the patient's throat and protects the esophageal walls during the next steps in the procedure.

The expanding assembly 120, preloaded with the endoscope 150 and the energy delivery assembly 160, (e.g., organ treatment assembly 100) is inserted into patient's body through the introducer 139. The expanding balloon is advanced distally positioning the shaped distal portion 215 against the distal side of the pyloric sphincter 22. Using an inflation fluid line, such as an air line, through a hand-piece 230 disposed at the proximal end of the organ treatment assembly, organ expanding member 140 is inflated (FIG. 4C) until the stomach's volume reaches the desired volume, such as at least about twice its empty volume (e.g. to about 2 liters). The pre-shaped distal portion 215, at the expanded configuration, seats against the distal side of the pyloric sphincter 22, providing an anchor and aiding in position and placement of the organ expanding assembly 120 within the organ (e.g., stomach). It should be appreciated that figures are for illustration purposes and are not intended to limit the scope of the present invention.

As explained earlier, the inflation fluid may be delivered through one of the endoscope lumens (or a tube extending along an interior thereof), through a lumen (or tube disposed therein) of the expanding elongate body, or through a separate fluid line extending along an exterior of the endoscope and within the expanding elongate body. In an embodiment, the organ's original volume, such as that of the stomach's, is increased by two fold, as for example from about 1 to about 2 liters.

Once the organ expanding member 140, 143 has been positioned at the treatment site and expanded to the desired volume, the endoscope 150 with the energy delivery member 166 disposed within, is pulled back proximally and navigated to the desired areas 196 within the organ. The endoscope is configured for flexible navigation and articulation within the organ.

In an embodiment, the practitioner using control unit 510 and foot pedal 511, introduces hot fluid, such as hot water from the hot fluid source 190, into the interior 203 of the energy delivery member 166 using the inflation/deflation lumen 151 of the endoscope or lumen 167 of the energy delivery elongate member. As hot water is introduced into the interior 203 of the energy delivery member 166, the energy delivery member 166 is extended out of the distal end of the endoscope 150 and into the interior 310 of the organ expanding member 140 which itself is located within the organ's interior 300. Under visual guidance by way of the endoscope 150, the expanded energy delivery member 166 is directed toward interior surface of the organ expanding member 140 corresponding to the desired treatment area/s 196 of the target organ, for directing energy (e.g., thermal energy by way of the hot fluid) to or the vicinities of the nerves, muscles and glands of the treatment zones 196. In an embodiment, the desired treatment area/s include any one or more areas corresponding to or in the vicinities of the greater curvature of the stomach, smaller curvature of the stomach, cardiac zone, gastric/fundic zone, pyloric zone, or the vagal nerve within the stomach.

In an embodiment, the expanded energy delivery member comes into direct surface contact with the desired surface area of the organ expanding member which corresponds to the desired target areas 196. Energy is delivered from the energy delivery member to the organ expanding member (FIG. 4D) which is in direct surface contact with the desired treatment sites 196.

In an embodiment, the transferred energy is sufficiently high to cause a change (e.g., physical, biochemical, physiological change) in the treatment target areas as described earlier.

It should be noted that hot fluid energy may be further augmented by delivery of energy to the targeted areas in the form of RF, other heat sources, microwaves, infrared or visible laser energy. For example, and as described in co-pending U.S. application Ser. No. 12/108,499 entitled Treating Medical Conditions of Hollow Organs, filed on Apr. 23, 2008 and assigned to the assignee of the present invention, the full disclosure of which is incorporated herein; the various assemblies may further be modified to allow the practitioner to activate an RF generator (not shown), resulting in additional energy being emitted from electrodes positionable in, on, or about the expanding member 140 to ablate the tissue in the treatment zones. During this time, using GUI 560 and feedback from corresponding sensor associated with such electrodes, the practitioner can watch for excessive temperatures. The duration of time and frequency of applied energy are, of course, responsive to judgments of medical personnel.

After the practitioner is satisfied that the desired amount of tissue has been treated (for example ablated) and/or the pulse transmissions between nerves and the brain have been affected by the desired amount, energy application is stopped. Energy delivery member 166 and organ expanding member 140 are deflated (FIG. 4E) and the energy delivery assembly 160 and organ expanding assembly 120 are withdrawn from the patient (FIG. 4F), as is the gastro introducer 139 (FIG. 4G).

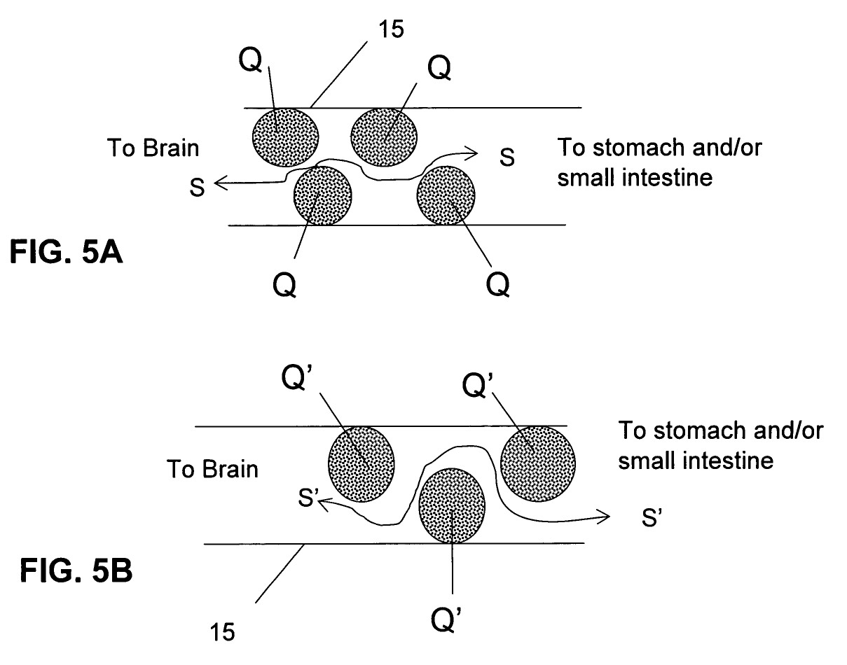

FIGS. 5A and 5B very schematically show the disruption and slowing of the travel of nerve pulses S, S’ between the stomach 12, the small intestine 14, and the brain. In FIG. 5A, smaller ablated portions Q of exemplary nerve 15 disrupt the straight flow of nerve signal impulses S between the stomach, small intestine, and brain. In FIG. 5B, larger ablated portions Q′ of exemplary nerve 15 more greatly disrupt the straight flow of nerve signal impulses S′ between the stomach, small intestine, and brain. The size of ablated portions Q, Q′ and the desired degree of associated signal disruption are left to the sound judgment of the practitioner after considering, for example, the degree of patient's obesity, strength of patient's hunger sensations, and variation in nerve size from patient to patient.

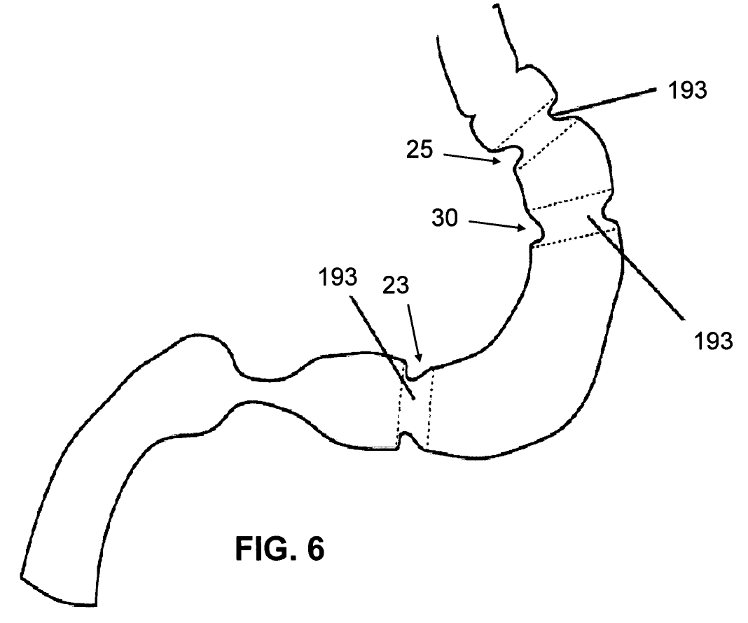

FIG. 6 is an exemplary depiction of the appearance of the muscle profile of a treated stomach about 3 months post-op. There will now be major muscular constrictions and lesions (dead tissue) in the areas of the fundus 25, peritoneum 30 and pylorus 23. These muscular constrictions and associated lesions should cause patient weight loss for the reasons discussed above. Because the procedure does not cause complete cell death in the treated areas, over long periods of time continued healing may cause the stomach's muscle profile to return to normal. Accordingly, follow-up treatments may be required. However, due to the process' simplicity, this should not pose any undue risk or inconvenience to the patient.

### Conclusion

While this application describes certain exemplary embodiments of treatments for weight-based medical conditions and apparatus useful for carry out the treatments, only the attached claims define the scope of the invention.

That which is claimed:

1. A treatment for medical conditions of hollow organs, comprising:
positioning a source of fluid thermal energy within the organ; and
applying thermal energy to at least one surface of the organ to affect the organ's operation.

2. A treatment according to claim 1, wherein the source of thermal energy comprises hot water.

3. A treatment according to claim 2, wherein the hollow organ is the stomach and the at least one surface of the stomach to which energy is applied is in the vicinity of at least a portion of a nerve communicating with the stomach and brain.

4. A treatment according to claim 1, wherein the hollow organ is the stomach and the at least one surface of the stomach to which energy is applied is a stomach muscle surface.

5. A treatment according to claim 4, further comprising applying energy to at least one surface of the stomach's underlying glands to affect glandular emissions.

6. A treatment according to claim 5, wherein the glandular emission is ghrelin.

7. A treatment for body-weight related medical conditions, comprising:
introducing an organ expanding member into a stomach;
expanding the stomach with the organ expanding member to expose any one or more of the underlying nerves, muscles, or glands of the stomach;
controllably introducing an expandable member adapted to deliver thermal energy into an interior of the organ expanding member; and
delivering thermal energy from the thermal delivery expandable member to a surface of the organ expanding member.

8. A treatment according to claim 7, wherein the thermal energy comprises hot fluid.

9. A treatment according to claim 7, wherein the at least one underlying muscle is in at least one of the greater curvature of the stomach, smaller curvature of the stomach, cardiac zone, gastric/fundic zone, or pyloric zone.

10. A treatment according to claim 7, wherein,
the organ expanding member comprises an expandable balloon having surfaces adapted for transferring energy to an inner surface of the organ; and
bringing into contact at least a portion of the surface of the energy delivery expandable member with at least a portion of energy transferring surface of the organ expanding member, whereby expanding the organ expanding member in the stomach positions the energy transferring surface of the organ expanding member in surface contact with at least one of the greater curvature of the stomach, smaller curvature of the stomach, cardiac zone, gastric/fundic zone, or pyloric zone.

11. A treatment according to claim 10, wherein a distal portion of the organ expanding member is pre-shaped to seat at least in part, upon expansion, in a distal side of a pyloric sphincter of the hollow organ.

12. A treatment according to claim 7, wherein the expansion of the organ expanding member within the organ, conforms the organ's interior to the organ expanding member.

13. An apparatus for treating medical conditions of hollow organs, comprising:
a hollow organ expanding member adapted for expanding in the hollow organ and exposing at least a portion of either or both the hollow organ's underlying nerves or muscle; and
an energy delivery expandable member disposable within an interior of the hollow organ expanding member and adapted to deliver thermal energy to the hollow organ using hot fluid as a source of thermal energy.

14. An apparatus according to claim 13, wherein the organ is the stomach and the organ expanding member is adapted to expand the stomach and includes surfaces for transferring energy to at least a portion of the stomach.

15. An apparatus according to claim 14, wherein the energy delivery member is expandable within the interior of the organ expanding member to deliver energy to at least a portion of the energy transferring surfaces of the organ expanding member.

16. An apparatus according to claim 15, wherein energy is transferred from the energy delivery member to the organ by way of direct surface contact with at least a portion of the energy transferring surface of the organ expanding member which is adapted for direct surface contact with at least one of the greater curvature of the stomach, smaller curvature of the stomach, cardiac zone, gastric/fundic zone, or pyloric zone.

17. An apparatus according to claim 16, wherein the energy transferring surfaces of the organ expanding member comprise visualization markings.

18. An apparatus according to claim 16, further comprising a visualization assembly for viewing the interior of the organ during a medical procedure.

19. An apparatus according to claim 13, wherein the organ expanding member is formed from expandable semi-compliant or non-compliant material.

20. An apparatus according to claim 13, wherein the organ expanding member is formed from a non-compliant material.
